(19) United States
(12) Patent Application Publication
(10) Pub. No.: US 2025/0241942 A1
(43) Pub. Date: Jul. 31, 2025

(54) METHODS OF SYNTHESIZING CONDUCTIVE POLYMERS IN BIOLOGICAL SYSTEMS AND METHODS OF USING CONDUCTIVE POLYMERS

(71) Applicant: Purdue Research Foundation, West Lafayette, IN (US)

(72) Inventors: Jianguo Mei, West Lafayette, IN (US); Sanket Samal, West Lafayette, IN (US); Krishna Jayant, West Lafayette, IN (US); Shulan Xiao, Lafayette, IN (US)

(21) Appl. No.: 19/040,515

(22) Filed: Jan. 29, 2025

Related U.S. Application Data
(60) Provisional application No. 63/626,508, filed on Jan. 29, 2024.

(51) Int. Cl. A61K 31/765; C08G 61/12

(52) U.S. Cl. CPC A61K 31/765; C08G 61/125; C08G 2261/11; C08G 2261/3242; C08G 2261/43; C08G 2261/514

(57) ABSTRACT

Methods of synthesizing biocompatible conductive polymers using endogenous catalysts, and methods of using such conductive polymers in biomedical applications. Such a method includes providing a biological system in which an endogenous catalyst is present, placing a quantity of 3,7-dihydrobenzo[1,2-b:4,5-b′]difuran-2,6-dione (BDF) monomer dissolved in a solvent into the biological system, and reacting the BDF monomer with the endogenous catalyst to synthesize n-doped poly(3,7-dihydrobenzo[1,2-b:4,5-b′]difuran-2,6-dione (n-PBDF) conductive polymer within the biological system.

### CROSS-REFERENCE TO RELATED APPLICATIONS

This application claims the benefit of U.S. Provisional Application No. 63/626,508 filed Jan. 29, 2024, the contents of which are incorporated herein by reference.

### BACKGROUND OF THE INVENTION

The present disclosure generally relates to the synthesis of conductive polymers, and particularly to the synthesis of conductive polymers within biological system utilizing a chemical compound present in the biological system as an endogenous catalyst for the polymerization process.

This section introduces aspects that may help facilitate a better understanding of the disclosure. Accordingly, these statements are to be read in this light and are not to be understood as admissions about what is or is not prior art.

Biocompatible conductive (conducting) polymers (CPs) have gained significant attention in biomedical applications due to their tunable electronic, optical, and electrochemical properties that can be utilized in various applications, as nonlimiting examples, bioimaging, drug delivery, biosensors, neural stimulation, and neural modulation. Traditionally, conductive polymers have been externally introduced into biological systems because they are not naturally produced in biological systems. As used herein, “biological systems” encompasses both nonliving organic systems and living systems (also referred to herein as living organisms). However, these strategies often result in poor bio-integration with soft tissues, creating a gap between in vitro and in vivo device performance and longevity.

To overcome such shortcomings, attempts have been made to synthesize conductive polymers within biological systems, with preliminary work showing that conductive polymers can be safely synthesized in vivo with electrochemical polymerization. Recent work has developed in vivo assembly of conductive polymers directly onto neural membranes, either by genetic engineering to express enzymes that catalyze polymerization or by using external oxidative enzymes that trigger endogenous metabolites (i.e., H₂O₂) to promote local polymerization in living fish and medicinal leeches. These methods still present limitations, such as the formation of toxic byproducts from over-expression of oxidative enzymes leading to cell apoptosis.

In view of the above, it would be desirable if processes existed by which conductive polymers could be assembled in vivo by only using endogenous metabolites to initiate and promote polymerization.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, methods of synthesizing biocompatible conductive polymers using endogenous catalysts, and methods of using such conductive polymers in biomedical applications.

According to a nonlimiting aspect, a method of synthesizing n-doped poly(3,7-dihydrobenzo[1,2-b:4,5-b′]difuran-2,6-dione (n-PBDF) conductive polymer includes providing a biological system in which an endogenous catalyst is present, placing a quantity of 3,7-dihydrobenzo[1,2-b:4,5-b′]difuran-2,6-dione (BDF) monomer dissolved in a solvent into the biological system, and reacting the BDF monomer with the endogenous catalyst to synthesize the n-PBDF conductive polymer within the biological system.

According to another nonlimiting aspect, a method of using a n-PBDF conductive polymer synthesized as described above includes using the n-PBDF conductive polymer in a biomedical application.

According to yet another nonlimiting aspect, a method is provided that includes synthesizing n-PBDF conductive polymer in vivo within a biological system by reacting BDF with an endogenous catalyst, and modulating neural excitability of neurons within the biological system using light-induced modulation of the n-PBDF conductive polymer.

Technical aspects of methods as described above preferably include the ability to synthesize conductive polymers in biological systems utilizing a chemical compound present in the biological systems as an endogenous catalyst for the polymerization process, such that the formation of toxic byproducts can be avoided.

These and other aspects, arrangements, features, and/or technical effects will become apparent upon detailed inspection of the drawings and the following description.

### BRIEF DESCRIPTION OF THE DRAWINGS

While some of the drawings shown herein may have been generated from scaled drawings or from photographs that are scalable, it is understood that such relative scaling within a drawing is by way of example, and are not to be construed as limiting.

FIGS. 1A through 1H relate to a biocompatible process for synthesizing a biocompatible polymer (n-doped poly(3,7-dihydrobenzo[1,2-b:4,5-b′]difuran-2,6-dione (n-PBDF)) in accordance with aspects of the invention. FIG. 1A represents a reaction scheme for the synthesis of n-PBDF. FIG. 1B represents endogenous hemeproteins used in investigations for oxidative polymerization of 3,7-dihydrobenzo[1,2-b:4,5-b′]difuran-2,6-dione (BDF) to synthesize n-PBDF. FIG. 1C is a graph comparing the UV-Vis-NIR (ultraviolet, visible and near infrared wavelength) spectrum of polymerization of BDF using hemin (“Hemin”) and copper acetate (“Copper Acetate”) to catalyze the synthesis of n-PBDF. FIGS. 1D and 1E relate to hemoglobin (Hb)-catalyzed polymerization of BDF to synthesize n-PBDF using different Hb concentrations, wherein FIG. 1D is a graph plotting data in the UV-Vis-NIR spectrum, and FIG. 1E is a graph plotting data in the DLS spectrum for hydrodynamic diameter. FIG. 1F is a bar graph plotting thin film conductivities of synthesized n-PBDF relative to the Hb concentration used to synthesize the n-PBDF. FIGS. 1G and 1H are graphs plotting hemoprotein-catalyzed polymerization of BDF to synthesize n-PBDF at different protein concentrations of myoglobin (“Mb”) (FIG. 1G) and cytochrome C (“Cyto-c”) (FIG. 1H).

FIGS. 2A through 2C are graphs relating to blood-catalyzed polymerization of BDF to synthesize n-PBDF, wherein FIG. 2A plots red blood cell-catalyzed polymerization of BDF to synthesize n-PBDF in different aqueous media, FIG. 2B plots whole blood-catalyzed polymerization of BDF to synthesize n-PBDF in different aqueous media, and FIG. 2C plots whole blood-catalyzed polymerization of BDF to synthesize n-PBDF at different BDF concentrations. FIG. 2D represents a proposed mechanism and catalytic cycle for the polymerization of BDF using blood to synthesize n-PBDF (the inset represents the EPR spectrum of ferryl Hb formation in the presence/absence of BDF and oxygen).

FIGS. 3A through 3H relate to the manner by which n-PBDF can be used for neural stimulation and neural modulation. FIG. 3A is a schematic representation of n-PBDF as a local “puff” near a patch clamped cortical pyramidal neuron in an acute brain slice. FIG. 3B represents components of the n-PBDF puff. FIG. 3C is an image showing the application of an n-PBDF puff near a cortical pyramidal neuron in an acute brain slice. FIG. 3D shows neural excitability reduced by an n-PBDF puff. FIG. 3E is a graph plotting firing rate as a function of current injection under a control condition and under a condition in which an n-PBDF puff is near a cortical pyramidal neuron in an acute brain slice (n=12 neurons, repeated measures ANOVA, p(1)<0.001). FIG. 3F is an image showing the application of PEDOT:PSS (poly(3,4-ethylenedioxythiophene) polystyrene sulfonate) as a local puff near a cortical pyramidal neuron in an acute brain slice. FIG. 3G shows neural excitability was not affected by a PEDOT:PSS puff. FIG. 3H is a graph plotting firing rate as a function of current injection under a control condition and under a condition in which a PEDOT:PSS puff is near a cortical pyramidal neuron in an acute brain slice (n=9 neurons, repeated measures ANOVA, p(1)<0.98).

FIGS. 4A through 4H relate to the manner by which light-induced modulation of neural excitability can be achieved via placement of n-PBDF. FIG. 4A is a schematic representation of an n-PBDF local puff applied to a dendrite of a patch clamped cortical pyramidal neuron in an acute brain slice (spaced 250 μm from the soma). FIG. 4B shows that an n-PBDF puff applied to a dendrite did not reduce neural excitability. FIG. 4C is a graph plotting firing rate as a function of current injection under a control condition and under a condition in which an n-PBDF puff is applied to a dendrite (n=4 neurons, repeated measures ANOVA, p(1)<0.99). FIG. 4D is a schematic representation of two-photon NIR spiral stimulation on an n-PBDF puff applied to a dendrite. FIG. 4E is a plot showing NIR light-evoked hyperpolarization and reversible neural excitability silencing (1040 nm, 6.6 mW, 1 s). FIG. 4F contains graphs plotting firing rates measured before, during, and after light stimulation in neurons under a control condition and under a condition in which an n-PBDF puff is applied to a dendrite (n=6 neurons for the n-PBDF puff condition and n=6 for the control condition, paired-sample t-test, *p<0.05, ***p<0.005). FIG. 4G is a plot showing the effect of laser power on firing rate change and inhibition of neuron activity. FIG. 4H is a graph plotting firing rate change as a function of laser power under a control condition and under a condition in which an n-PBDF puff is applied to a dendrite (n=6 neurons for the n-PBDF puff condition and n=6 for the control condition).

FIGS. 5A through 5I relate to in vivo polymerization of BDF to synthesize n-PBDF and in vivo neuromodulation achieved via n-PBDF. FIG. 5A represents polymerization of BDF using whole blood to catalyze the synthesis of n-PBDF in live zebrafish embryos at 34° C. and after 24 h incubation. FIGS. 5B and 5C contain microscopic images of zebrafish embryos with darkened yolks without 1-phenyl 2-thiourea (PTU) treatment (FIG. 5B) and with PTU treatment (FIG. 5C). FIG. 5D is a schematic representation of NIR absorption of live zebrafish embryos with a 960 nm LASER focused on the yolks of the embryos. FIG. 5E contains NIR absorption images of live zebrafish embryo yolks with 15 mM BDF injection after incubation in comparison to no BDF injection. FIG. 5F is a bar graph evidencing the viability of live zebrafish embryos with darkened yolk in relation to increase injected BDF concentrations. FIG. 5G is a schematic representation of recording neural activities in an awake mouse with n-PBDF synthesized in vivo. FIG. 5H contains images representing experimental setups with a silicon probe recording in Ml under local injection of a control buffer (top image) and n-PBDF synthesized in vivo (bottom image). FIG. 5I is a graph showing that n-PBDF synthesized in vivo increased the inter-spike-interval (ISI) (n=102 neurons from 3 mice, Wilcoxon signed rank test, p<0.001).

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawing. The following detailed description also describes certain investigations relating to the embodiment(s) and identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments. Therefore, the appended claims, and not the detailed description, are intended to recite particularly point out subject matter regarded to be aspects of the invention, including certain but not necessarily all of the aspects and alternatives described in the detailed description.

The following describes processes and investigations by which biocompatible conductive polymers can be synthesized within a living system (in vivo) from a monomer delivered into a biological system and polymerized with an endogenous catalyst for the polymerization process that is present in the biological system. The ability to synthesize biocompatible conductive polymers utilizing catalysts available within a living system offers possibilities for a variety of biomedical applications, as nonlimiting examples, bioimaging, drug delivery, biosensors, neural stimulation, and neural modulation.

The investigations described below included the in vivo synthesis of a biocompatible n-doped conductive polymer (n-doped poly(3,7-dihydrobenzo[1,2-b:4,5-b′]difuran-2,6-dione; n-PBDF) within live zebrafish embryos, achieved through catalyzed polymerization of 3,7-dihydrobenzo[1,2-b:4,5-b′]difuran-2,6-dione (BDF) with endogenous catalysts. As described in greater detail below, the efficacy of such a polymerization was rigorously established through a sequence of in vitro experiments involving hemin, hemoproteins, red blood cells, and whole blood as the endogenous catalyst. The polymerization approach was shown to demonstrate exceptional resilience under biological conditions and serve as a highly robust synthetic method. Furthermore, the non-toxicity of the polymerization process and the resulting polymer was confirmed by subcutaneously administering BDF and n-PBDF solutions in mice. No physical stress or discomfort was observed in the mice, and the complete blood count (CBC) demonstrated no signs of acute toxicity except for a minor level of hemolysis. The investigations reported below explored the use of n-PBDF to modulate the neural activity of cortical pyramidal neurons in mice acute brain slices, showing the interaction of n-PBDF with the neurons at a cellular level, leading to a decrease in the intrinsic excitability of the neurons. The investigations showed that, when n-PBDF was moved from the soma to the dendrites of neurons, the neurons were capable of producing strong hyperpolarization induced by the NIR stimulation and can reversibly modulate neuronal activity. The investigations also confirmed the ability to form n-PBDF in vivo within living systems (live zebrafish embryos) without causing any fatalities, demonstrating excellent biocompatibility. Additionally, in vivo modulation of neural activity was confirmed in awake head-fixed locomoting mice using n-PBDF, exhibiting decreased electrical activity. These and other aspects of the method will be more fully appreciated from the following discussion of the investigations.

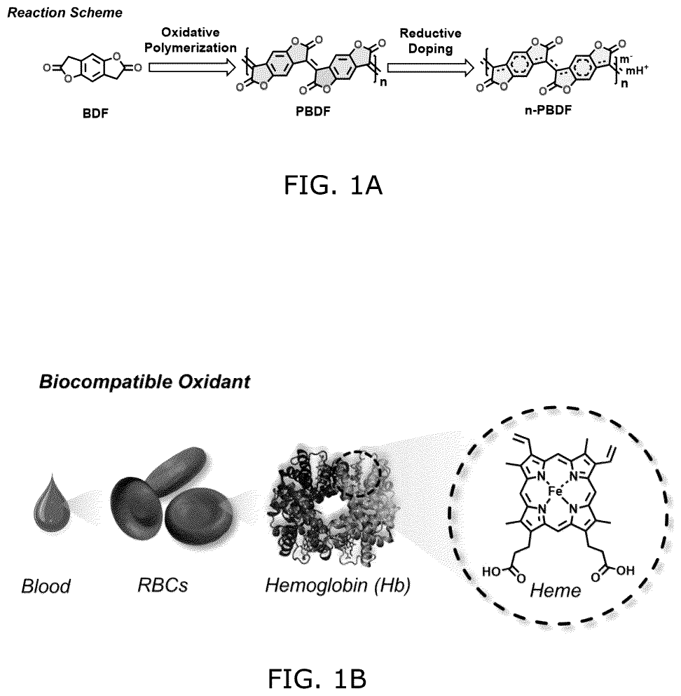

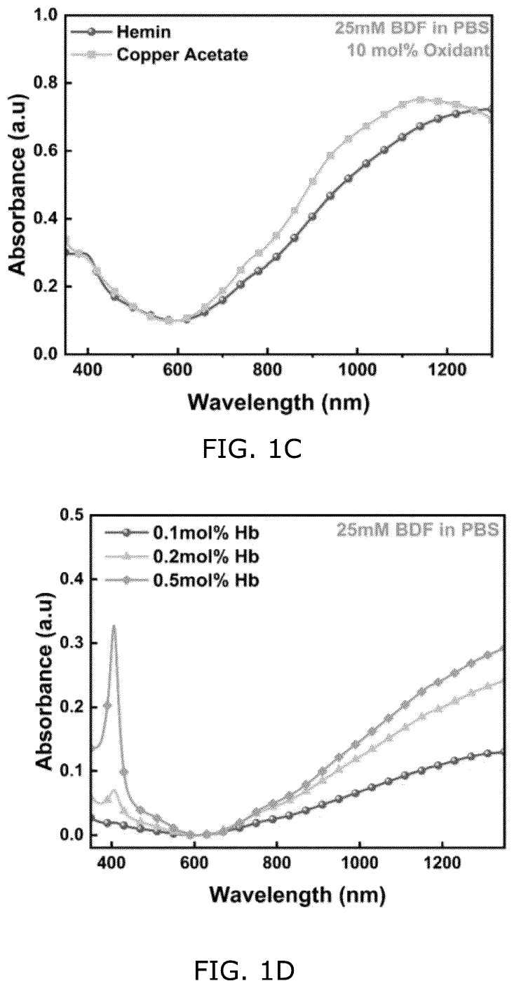

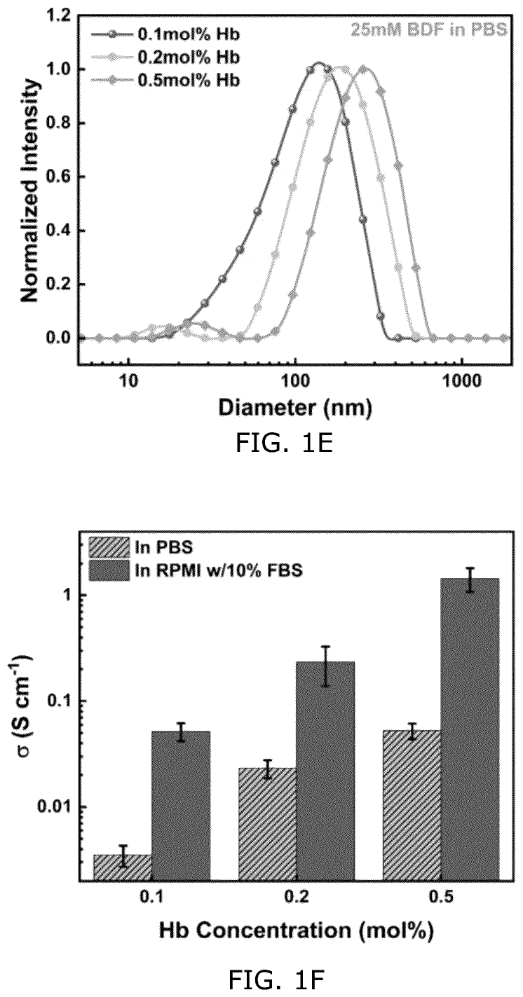

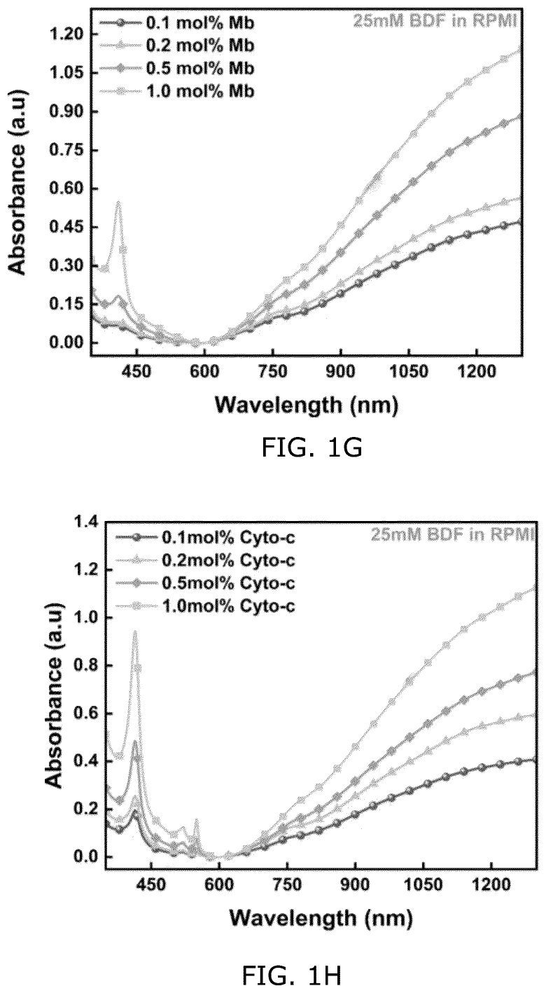

n-PBDF has been reported to show features such as high conductivity, air/water stability, and biocompatibility. FIG. 1A represents a reaction mechanism for synthesizing n-PBDF that involves oxidative polymerization of BDF by mild oxidants and reductive doping by water. The investigations leading to the present invention discovered that utilizing endogenous enzymatic proteins, for example, hemin, hemoproteins, red blood cells, and whole blood (FIG. 1B) can lead to efficacious in vivo polymerization of BDF to synthesize n-PBDF in aqueous media. Included in the definition of hemoproteins are hemoglobin (found in blood), myoglobin (found in muscle tissue), cytochrome (found in mitochondria of various cells), catalase (found in peroxisomes of various cells), and peroxidase (found in animals, plants, and fungi.

Though existing methods of synthesizing n-PBDF are efficient, they are unsuitable within living systems. The BDF monomer has limited solubility in organic solvents and is insoluble in aqueous media. Existing synthesis methods also require high temperatures and employ dimethyl sulfoxide (DMSO) as a solvent. To overcome this limitation, methods were investigated for synthesizing n-PBDF in aqueous media using surfactants, which are also utilized in the synthesis of conductive polymer nanoparticles. A promising option was found to be vitamin E-based TPGS-750-M (tocopheryl polyethylene glycol succinate). Using TPGS as a surfactant, n-PBDF can be polymerized in aqueous media through emulsion polymerization. By incorporating an insignificant amount (5%) of DMSO and 1% w/w TPGS, this method was found suitable for use in biological systems, as discussed below.

To verify the effectiveness of this synthesis approach, the polymerization of BDF to synthesize n-PBDF in an aqueous 1× phosphate buffered saline (PBS) (pH 7.4) media solution at human body temperature (37° C.) was investigated using copper acetate as the catalyst with and without the surfactant. The polymerization method was concluded to be highly consistent, resulting in an aqueous n-PBDF ink that was analyzed using a UV-Vis-NIR spectrophotometer, showing strong absorption in the NIR region with the use of TPGS surfactant and matches reported results (FIG. 1C). In addition, TPGS-750-M's effectiveness was compared to other surfactants, such as Triton X-100, which is widely used in biological systems for immunostaining and DNA extraction. The polymerization process was found to be more efficient with TPGS-750-M than with Triton X-100, yielding a highly doped polymer using a smaller amount of surfactant. Although copper is highly competent as a catalyst, it is found in trace quantities in living systems, and excessive levels of copper are toxic, making it unsuitable as a catalyst for in vivo polymerization applications. To increase the viability of the polymerization process, endogenous iron-based catalysts were investigated, as iron is essential in biological systems and could facilitate redox reactions, making it the fundamental component for many bio-enzymes and proteins.

The ability of iron to catalyze the polymerization of BDF to synthesize n-PBDF was evaluated by examining hemin as a potential source of iron. Hemin contains a ferric ion and a coordinating chloride ligand resembling the critical components of many hemoproteins. It was determined that 10 mol % hemin was successfully able to catalyze the polymerization of BDF to synthesize n-PBDF (FIG. 1C). However, further investigation indicated that the doping level of n-PBDF decreased as the amount of hemin was increased from 10 mol % to 100 mol %, likely due to the free Fe³⁺ ion in the solution that de-dopes the polymer and results in it precipitating out from the n-PBDF polymer ink. This observation opened a potential pathway for using heme-containing bioactive proteins to facilitate in vivo polymerization of BDF to synthesize n-PBDF. Of all hemoproteins, hemoglobin (Hb) is the most widely recognized as it plays the crucial role of transporting oxygen in the vascular system of animals. Investigating the use of Hb as a catalyst, it was observed that considerable polymerization occurred with only 0.1 mol % of Hb, causing the solution to turn black within twenty minutes. By increasing the Hb amount from 0.1 mol % to 0.5 mol %, an improvement was observed in the doping level of n-PBDF, along with a higher conversion rate (FIG. 1D). Furthermore, using dynamic light scattering (DLS) measurements, it was observed that the hydrodynamic diameter of the n-PBDF particles increased from 130 nm to 264 nm as the concentration of Hb was raised from 0.1 mol % to 0.5 mol % (FIG. 1E), indicating a higher conversion as the Hb concentration was increased. In addition, when the reaction was performed in an oxygen environment, the catalyst's reactivity increased, giving higher conversion and doping levels.

To mimic in vivo conditions, the PBS media solution was replaced by an aqueous media solution containing RPMI 1640 (also known as RPMI medium), which is well known as a cell culture medium used to culture mammalian cells. The media solution further contained 10% fetal bovine serum (FBS) and 1% penicillin-streptomycin. It was observed that polymerization was more effectual in the RPMI-containing media solution than in the PBS media solution. A comprehensive kinetic study was conducted using UV-Vis-NIR measurements to assess the effects of switching solvents from PBS media solution to the RPMI-containing media solution. It was observed that the reaction occurred faster in the RPMI-containing media solution compared to the PBS media solution while maintaining the same concentrations of BDF and Hb. The RPMI-containing media solution with FBS most likely contains some Hb and other hemoproteins, leading to better conversion and higher doping levels within a six-hour reaction interval compared to the PBS media solution. The conductivity measurements of n-PBDF thin films were consistent with those of polymers synthesized in different mediums (FIG. 1F). Regardless of the Hb concentration, it was observed a notable rise in conductivity when changing the medium from the PBS media solution to the RPMI-containing media solution, achieving a conductivity of 1.6 S cm⁻¹. This level of conductivity was comparable to other conducting materials that are directly assembled in living systems and matches the conductivity requirements for various biomedical applications. It was noted that the presence of insulating hemoglobin and a surfactant in the thin films might lead to an underestimation of conductivity.

To investigate the universal nature of the heme-containing catalytic system, various other hemoproteins were analyzed, such as myoglobin (Mb) and cytochrome C (Cyto-c). Through screening, it was observed that regardless of the type of protein utilized, comparable outcomes were observed with increased conversion and higher doping levels when the quantity of protein was increased. It was observed that efficient polymerization of BDF to synthesize n-PBDF occurred when at least 0.1 mol % of the hemoprotein was present in the reaction media (FIGS. 1G and 1H). This amount was comparable to the quantity of Hb found in a single drop of blood from a healthy adult female. However, Hb was the fastest and most efficient because it possesses four heme groups, a distinct advantage over Mb and Cyto-c, which only contain one.

To delve deeper into the impact of BDF concentration on its polymerization, the BDF concentration was altered from 10 mM to 50 mM while maintaining a steady Hb concentration of 25 μM. It was observed that irrespective of the BDF concentrations, the polymerization rate remained the same. For a BDF concentration of 10 mM, complete conversion was observed after 2 hours, while for higher BDF concentrations of 25 mM and 50 mM, the reaction continued for 6 hours and then gradually slowed down. This phenomenon was attributed to the slow, gradual iron release from the heme core, leading to the decomposition of Hb. As a result, the intensity of the Hb peak (410 nm, specific to Fe³⁺-heme) decreased and eventually disappeared after about six hours. Furthermore, to better comprehend the catalytic effect of Hb, the Hb concentration was altered from 0.1 mol % to 0.5 mol % while maintaining a constant BDF concentration of 25 mM. It was observed that the polymerization rate increases as the concentration of the Hb increases. Hence, it can be inferred from these observations that the enzymatic polymerization of BDF to synthesize n-PBDF using hemoproteins follows zero-order enzyme kinetics. However, after twelve hours of reaction, a decrease in doping levels was observed, which was more significant in the case of 0.5 mol % of Hb concentration. When considering the 0.2 mol % of Hb concentration, there was still a noticeable reduction in doping levels. This was consistent with the observations in the case of hemin, as the Hb slowly degrades, producing free Fe³⁺ ions, which can de-dope the n-PBDF.

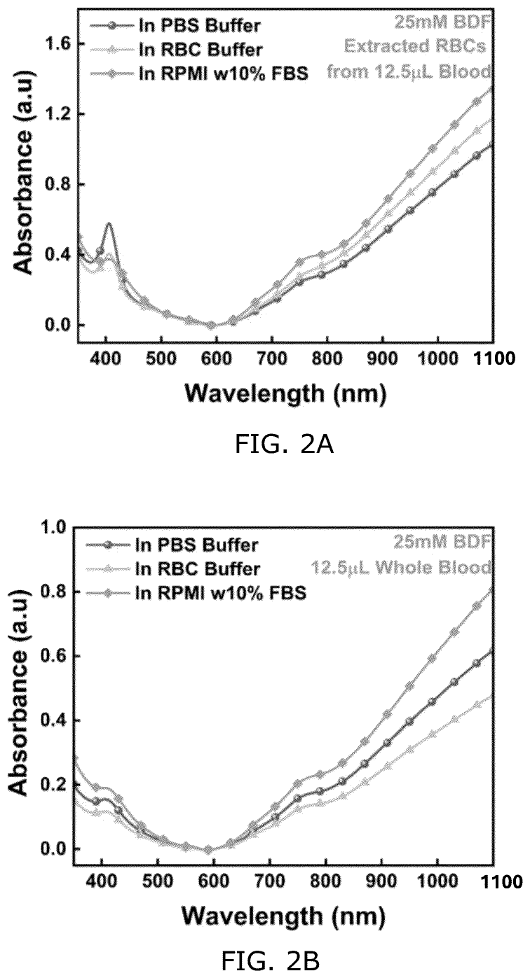

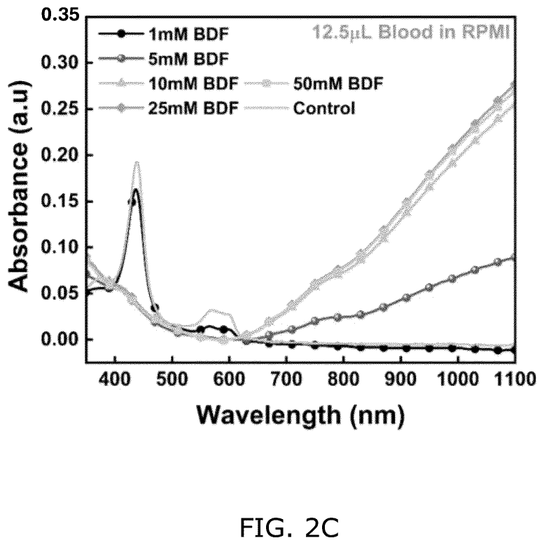

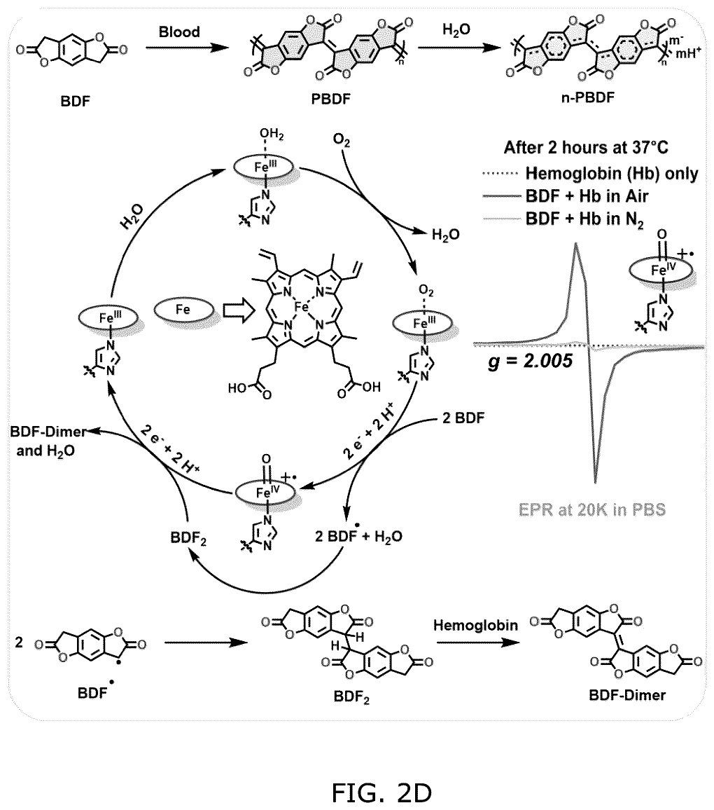

After successfully polymerizing n-PBDF using lyophilized hemoproteins, an investigation was conducted to assess the potential for naturally occurring Hb in red blood cells (RBCs) to catalyze the oxidative polymerization of BDF to synthesize n-PBDF. Upon comparing isolated lyophilized bovine Hb powder and freshly isolated RBCs (erythrocytes) from female human blood, it was observed that there was hardly any noticeable difference between the two, with similar conversion and reaction rates in a RPMI-containing media solution (FIG. 2A). Using freshly obtained human female whole blood as the catalyst yielded a significantly higher conversion and doping level than using isolated RBCs (FIG. 2B), proving the robustness of the catalytic system. After conducting an investigation into the polymerization of BDF to synthesize n-PBDF with whole blood, it was observed that a minimum concentration of 5 mM BDF was necessary for n-PBDF formation when BDF concentration was varied from 0.1 mM to 50 mM (FIG. 2C) while using 12.5 μL of whole blood. Additionally, a slight decrease in the doping level of the n-PBDF polymer was observed when the quantity of whole blood was increased. This result was consistent with previous findings when utilizing lyophilized Hb as the catalyst.

To evaluate the efficacy of polymerization without the need for constant stirring, a method was employed whereby the solution was placed in an incubator with a rocker at 37° C. Upon introduction of whole blood into the reaction vessel without stirring, a sluggish reaction was observed, and the vessel turned black after three hours into the reaction. Additionally, it was observed that the conversion rate was lower in a closed-cap vessel but improved significantly in the presence of an air balloon. Based on these observations from the reaction without stirring, it was deduced the lysis of the RBCs with the BDF monomer. To confirm, a hemolysis assay was used on the various reaction components and with different BDF concentrations. It was found that 5% v/v DMSO and 1% w/w TPGS caused less than 1% of red blood cells to lyse. The BDF monomer resulted in significant hemolysis of approximately 40% at 0.2 mg/mL. This explained the initial slow reaction and formation of n-PBDF without stirring.

To investigate the mechanism of n-PBDF formation using hemoproteins, Mb and 2-coumaranone (BF) were selected as a model system. When Mb was mixed with BF at 37° C. (human body temperature) with and without surfactants, the rise of two prominent peaks was observed at 542 nm and 580 nm corresponding to the ferryl Mb. Furthermore, these ferryl Mb peaks only occurred in the presence of either BF or BDF and oxygen. To confirm the formation of ferryl Mb, Raman measurements of Mb were conducted in the presence of BF at 37° C. A decrease was observed in the peak intensity at 1560 cm⁻¹ corresponding to the Fe³⁺-Mb and the rise of a tiny peak at 780 cm⁻¹ corresponding to the ferryl Mb. However, the peak intensities corresponding to the ferryl Mb were weak, leading to a poor signal-to-noise ratio. Therefore, low-temperature EPR measurements of the n-PBDF polymerization were explored using Hb in the PBS media solution. When only Hb was heated at 37° C. for 2 hours, it produced no EPR signals. However, in the presence of the BDF monomer, it gave a strong peak at g=2.005, corresponding to the formation of ferryl Hb. As the polymerization progressed, the intensity of the ferryl Hb peak increased, which was distinguishable from the EPR peak of the n-PBDF polaron. Furthermore, polymerization was prevented when the reaction was carried out in a nitrogen atmosphere, and the EPR peak for the formation of ferryl Hb was almost negligible. To verify the importance of ferryl Hb in polymerization, n-PBDF polymerization using Hb was conducted in the presence of ascorbate (vitamin C), an antioxidant known to reduce ferryl Hb to low oxidation states. Polymerization was strongly inhibited as ascorbate amounts were increased from 10 mol % to 100 mol %, characterized by a decrease in UV-Vis-NIR absorption. To verify the radical pathway, polymerization was conducted in the presence of radical scavengers like (2,2,6,6-tetramethylpiperidin-1-yl)oxyl (TEMPO) and N-tert-butyl-a-phenylnitrone (PBN) under a nitrogen environment. In the case of TEMPO, the polymerization, although slow due to the nitrogen atmosphere, was not inhibited due to TEMPO's oxidative properties, which have been reported to help in the oxidative polymerization of PBDF. On the other hand, in the case of PBN, polymerization was substantially prevented, with no color change observed in the reaction vessel. After six hours, almost no polymer was observed via the UV-Vis-NIR spectroscopy. On this basis, FIG. 2D represents a proposed radical-based mechanism and the catalytic cycle.

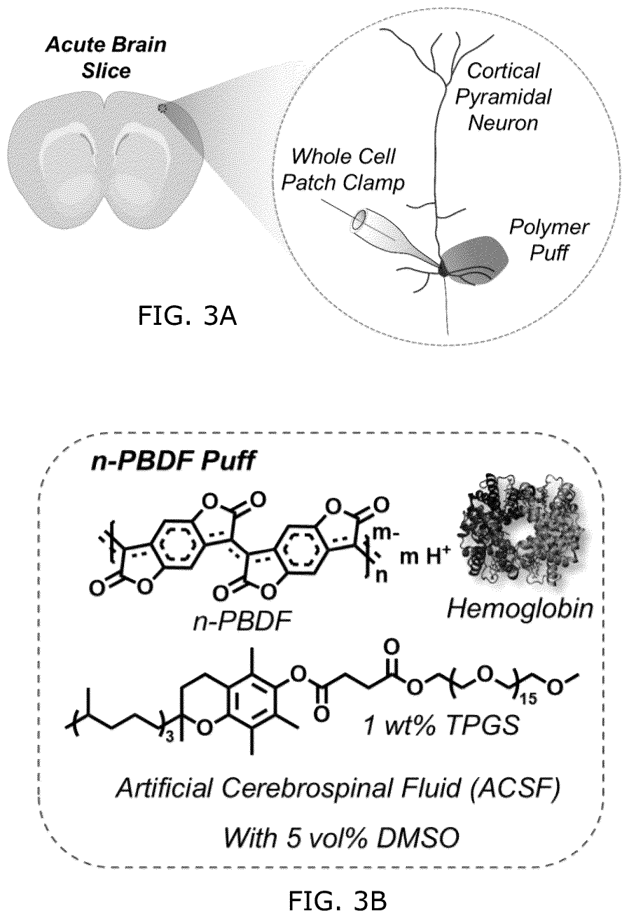

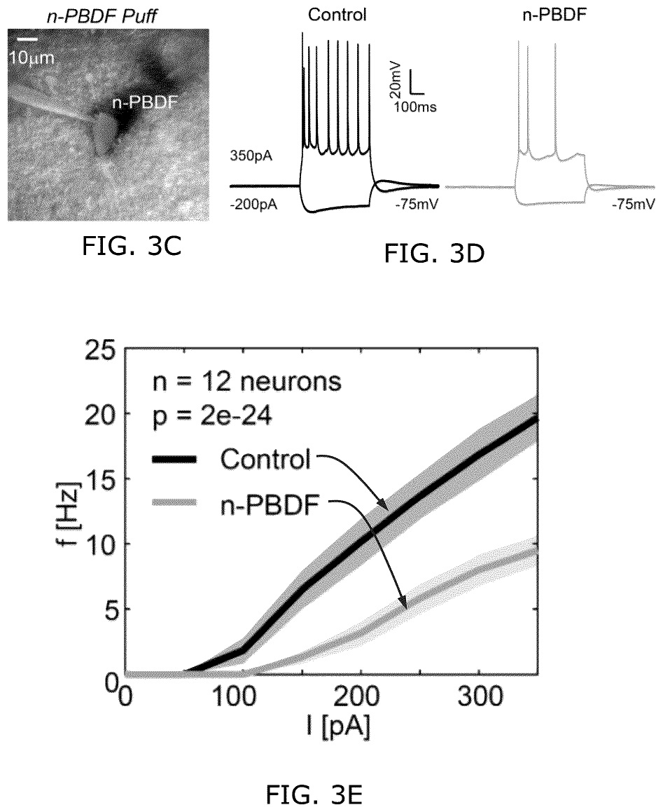

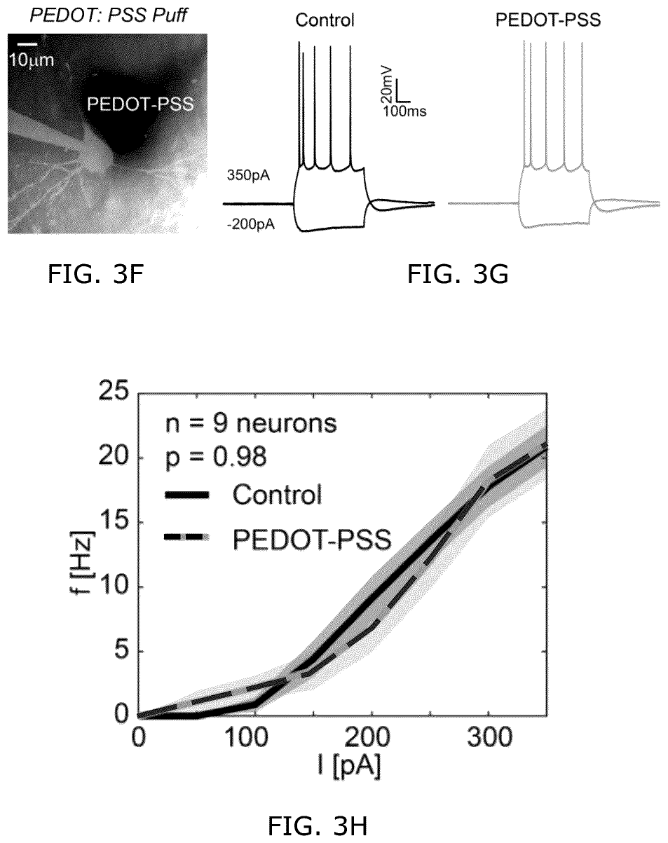

The effect of n-PBDF on electrophysiological behaviors of layer 5 cortical pyramidal neurons in acute brain slices of mice was further examined (FIG. 3A). Previous studies had shown that the in vivo assembled p-type conductive polymers that integrate themselves within the neuronal membrane are known to change the membrane capacitance, leading to a decrease in neuronal excitability. However, the effect of the n-type conductive polymers injected around the neuronal membrane does not appear to have been previously reported. The impact of pipette-injected n-PBDF on the neuronal activity of the pyramidal neurons was characterized using whole-cell patch clamp recordings. A small amount of an aqueous solution of n-PBDF synthesized using Hb as a catalyst was injected in artificial cerebrospinal fluid (ACSF), with a BDF concentration of 10 mM, around the patched pyramidal neuron (FIG. 3B). Although a slight decrease in membrane resistance was observed after the n-PBDF injection, no statistically significant changes in membrane capacitance were detected. The effect of pipette-injected n-PBDF on the intrinsic neuronal excitability was examined using spike frequency-current relationship (F-I curves) and a reduction of action potential (AP) firing under depolarizing stimuli was observed (FIGS. 3C through 3E, n=12 neurons, repeated measures ANOVA, p<0.001). Significant changes in the AP amplitude, AP width, or leak current were not observed, suggesting no adverse impact on the neuron's intrinsic properties after n-PBDF injection, and the patched cells remained healthy during further testing under all conditions. To investigate whether the observed decrease in the intrinsic excitability of the neurons was a unique property of the n-PBDF or if it was due to the conductive nature of the conductive polymers, a small amount of an aqueous solution of PEDOT:PSS diluted in ACSF (to achieve a similar concentration and composition as n-PBDF) was injected (FIG. 3F). No significant change in membrane resistance and membrane capacitance was observed after the PEDOT:PSS injection, and the neurons exhibited no changes in the nature of AP firing to depolarizing stimuli (FIGS. 3G and 3H, n=9 neurons, repeated measures ANOVA, p<0.98). While in situ assembled p-type conductive polymers have been shown to create an inverse correlation between spike firing and membrane capacitance when conductive polymers are inserted into the neuronal membrane, this mechanism was not observed but instead pointed to a resistive effect, suggesting a different mechanism at play.

To gain a deeper understanding of the mechanism that resulted in a significant decrease in the inherent excitability of the neurons at a cellular level, the interaction of n-PBDF with pyramidal neurons was explored by measuring and characterizing the transmembrane ionic currents. As the n-PBDF solution was synthesized in Na⁺-rich ACSF, it was hypothesized that the presence of a high concentration of Na⁺ ions (as the counter ion in n-PBDF) could influence ionic concentration gradients locally and, in turn, impact ion-channel gating characteristics, resulting in modulation of activity. To measure this, an initial experiment was conducted to assess the impact of n-PBDF injection on the Na⁺ and K⁺ currents. An increase was observed in Na⁺ current and a decrease in the outward K⁺ current. In contrast, no changes were observed in the Na⁺ and K⁺ currents with PEDOT:PSS injection. This observation could be related to the high local concentration of the Na⁺ ions present in the n-PBDF injected around the neuronal membrane, which could lead to an increase in inward Na⁺ current while reducing the outward K⁺ current. Such ionic clouds could, in principle, lead to different gradients locally and gate ion channels. PEDOT:PSS injection, on the contrary, did not show a similar effect, which could be due to the absence of the Na⁺ counter ion, as found in n-PBDF. The gating properties of Na⁺ and K⁺ channels were further investigated. Despite an increase in the maximum Na⁺ channel conductance, the steady-state characteristics and trend of activation and inactivation of Na⁺ channels remained intact after n-PBDF was locally injected. This matches the observation in which an increase in the inward Na⁺ current was found. A significant reduction was observed in the rate of Na⁺ ion channel recovery from inactivation (τ increases from 6.88 ms to 11.98 ms; n=4 neurons, repeated measures ANOVA, p<0.03), as well as the outward K⁺ channel conductance (n=5 neurons, repeated measures ANOVA, p<0.001). Collectively, these results suggested a model in which n-PBDF modulates membrane excitability via ionic gating and intrinsic property changes, mainly recovery from inactivation and active conductance. Such a non-genetic mechanism of neuromodulation impacting the temporal properties of channels could be utilized to control neural rate and time coding.

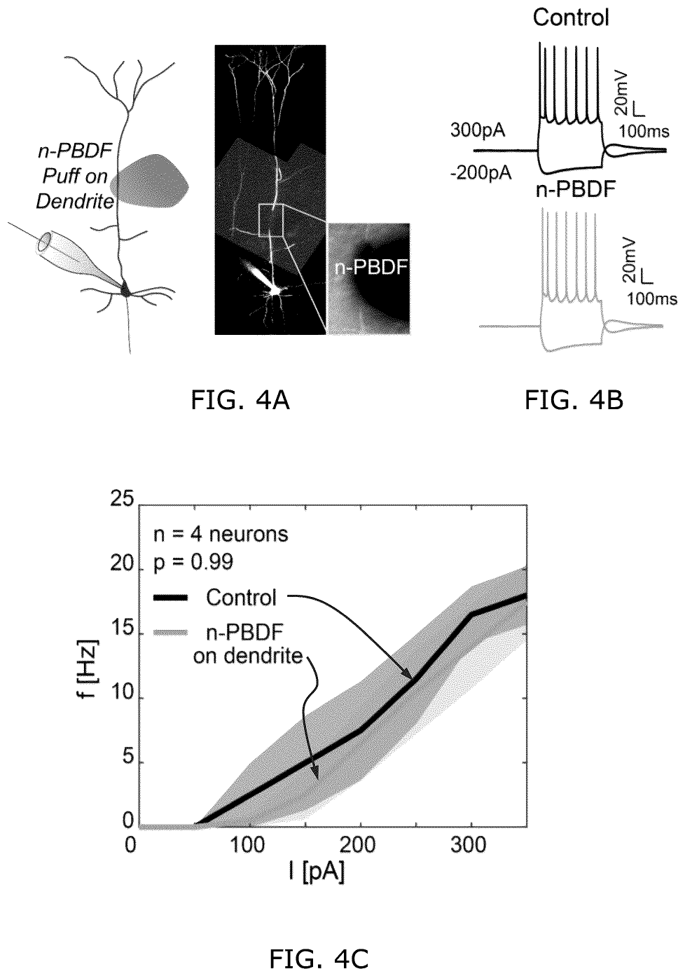

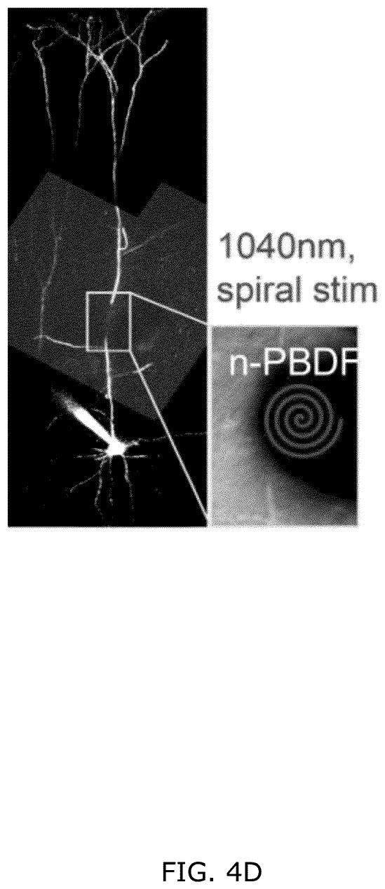

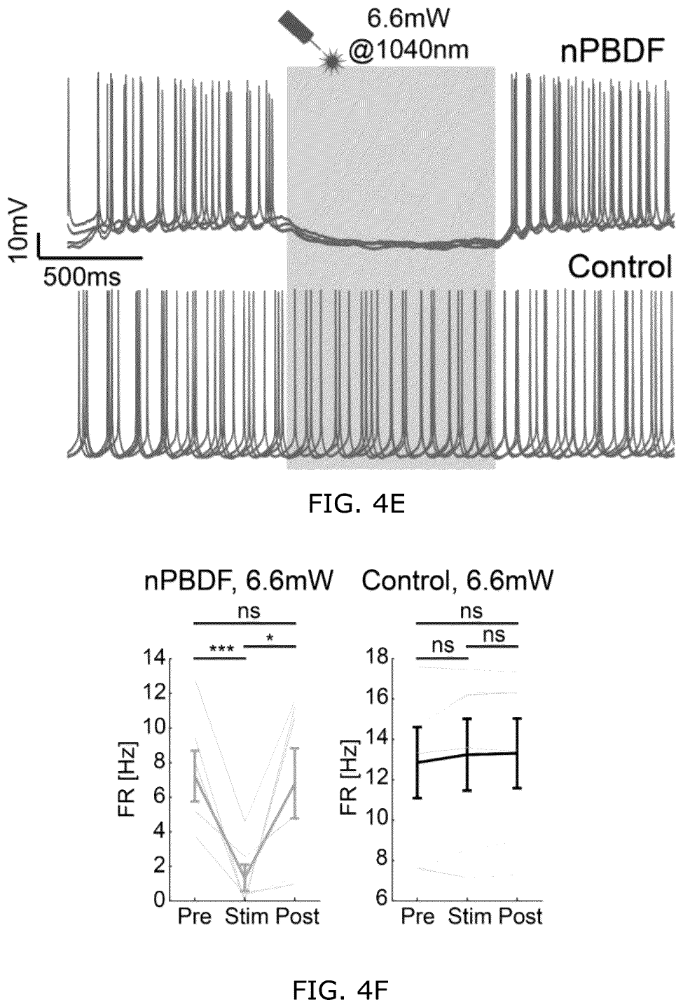

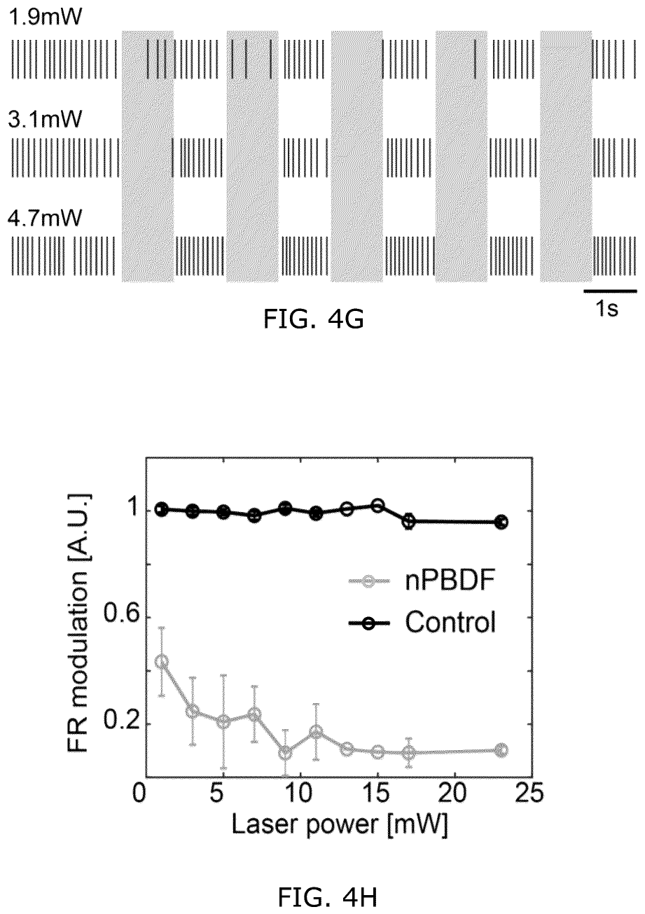

Native n-PBDF locally injected near the soma of cortical pyramidal neurons resulted in reduced AP firing. While this was of interest and could open a pathway to suppress the intrinsic excitability of neurons, it was considered as to whether this form of neuromodulation could be controlled effectively and applicable across different segments of a neuron. To this end, n-PBDF was injected on the apical dendrite of cortical pyramidal neurons (approximately 250 μm away from the soma), avoiding the localized increase in Na⁺ ion concentration around the soma and axon initial segment (AIS), where the Na⁺ channels are dense (FIG. 4A). These apical dendrites play a critical role in neural computation and learning and potently control neuronal gain, yet targeted/focused modulation of dendritic excitability is limited using currently available techniques, including genetic engineering optogenetics as there is no opsin currently that can selectively target dendrites. Previous reports have indicated NIR-induced modulation of neural activity in primary hippocampal neurons plated onto poly(3-hexylthiophene) (P3HT) thin film, due to the change in the reversal potential induced by local temperature change. Given the strong light absorption of n-PBDF in the NIR region (FIG. 1D) and the effect of n-PBDF on ion channel gating, it was hypothesized that the NIR photo-stimulation of n-PBDF can be used as an additional control to tune the modulatory effect of n-PBDF on neuronal excitability. However, previous reports have indicated that single-photon photo-stimulation alone in vivo can induce changes in rectifier potassium channel properties via heating. Therefore, two-photon NIR stimulation was investigated to localize the stimulation effect on locally injected n-PBDF near the apical dendrites and assay its role in controlling dendritic excitability. In contrast to the perisomatic injection of n-PBDF, a similar decrease in cellular excitability was not observed when the n-PBDF was injected near the apical dendrite without photo-stimulation (FIGS. 4B and 4C). A1040 nm femtosecond pulse laser was then used to perform spiral stimulation on the injected n-PBDF without directly scanning onto the main apical dendrite (FIG. 4D). Somatic current injection was applied to elicit firing in the targeted neuron. With laser stimulation (max scan time of 1 s; min 10 ms, 6.6 mW) on n-PBDF, spiking was suppressed completely but reversed immediately upon removal of the spiral scan (FIG. 4E). This suppression effect was not observed in neurons without n-PBDF under the same NIR light stimulation (FIG. 4E, lower plot). The firing rate was significantly reduced during NIR stimulation (1 s, 6.6 mW) in the presence of n-PBDF, and the reduction in spiking was absent without n-PBDF. (FIG. 4F, n=6 neurons with n-PBDF and 6 without, paired-sample t-test, *p<0.05, ***p<0.005). It is important to note that scan times of about 1 s are higher than typically used for two-photon optogenetics (on the level of 1 to 10 s of ms). However, prolonged two-photon stimulation is not uncommon in two-photon optogenetic inhibition since it is nearly impractical to detect the onset of every spike and suppress with short light pulses in a close-loop manner, especially when a complete suppression over a prolonged time window is desired. No changes were observed in the baseline firing rate (FIG. 4F) or the resting membrane potential before and after the NIR stimulation (1 s, 6.6 mW), suggesting minimal photodamage induced by NIR stimulation. The laser power required for the spike suppression under NIR stimulation on n-PBDF was further characterized (FIG. 4G). An approximately 50% decrease in AP firing was observed with laser power as low as 1.9 mW and complete silencing at higher laser powers (FIG. 4H). Compared to typical two-photon optogenetics (about 20 mW), the laser power used (as low as 1.9 mW and no more than 10 mW for a complete suppression) was much lower.

Previous studies suggest that thermal heating can suppress neural activities. With high IR absorption of n-PBDF, the generation of photo-excited states that recombine non-radiatively to the ground state can result in the local release of thermal energy. Therefore, it was hypothesize that photothermal properties, in addition to the modulation of ion channels by n-PBDF, could explain the NIR-induced suppression of spiking. To corroborate this hypothesis, the local temperature change was measured with two-photon NIR stimulation on n-PBDF injected into acute brain slices. A rapid increase in temperature was observed at the onset of the NIR light stimulation, which persisted throughout the illumination period. The temperature recalibrated to baseline after the light stimulation ceased. The temperature change depended on the laser power, yet a low-power NIR stimulation at 1.9 mW could lead to an approximately 10° C. increase in temperature in the presence of n-PBDF. In contrast, the change in temperature without n-PBDF was almost negligible under the comparable power of NIR stimulation and only achieved a similar increase of approximately 10° C. with laser powers>150 mW. Notably, a clear hyperpolarization was observed when a short NIR light pulse of 50 ms was applied to the neuron without external current (RMP approximately −60 to −55 mV). This suggested an outward current effect, possibly due to the thermal effect on potassium channels. Furthermore, the hyperpolarization amplitude increased with increasing laser power. This hyperpolarization was absent without the n-PBDF injection, suggesting the photothermal properties of the polymer could be critical for this hyperpolarization. To further verify the role of n-PBDF in the suppression of spiking, NIR stimulation was performed near apical dendrite in the absence of n-PBDF to test the effect of photo-stimulation alone on spiking. To create a similar temperature change of about 10° C., laser power as high as 150 mW was used. However, the high-power NIR stimulation alone slightly suppressed the spiking (7.2% decrease, 150 mW) but not to the same extent in the presence of n-PBDF at comparable temperature change (56.6% decrease, 1.9 mW). Furthermore, a rebound excitation was observed after photo-stimulation removal without n-PBDF, which could counteract the intended activity suppression. This suggested that the thermal effect alone did not fully explain the extent of modulation, and the intrinsic properties of n-PBDF, such as strong NIR absorption and ion channel gating modulation, could play a role. It is worth noting that although no change was observed in the apparent electrical properties (firing rate, RMP—FIG. 4F) and cell morphology (FIG. 4A) before and after the photo-stimulation, as described earlier, caveats of this photothermal modulatory approach may exist due to the sudden heat shock induced by the NIR stimulation on n-PBDF. Previous reports indicated that heat shock may induce changes in the internal signaling of neurons, such as Ca²⁺ storage release and synaptic transmission. The investigation results suggested the potential of using n-PBDF for non-invasive neural stimulation and modulation through NIR stimulation without needing genetic modification, with its high biocompatibility, modulatory capability of neuronal excitability, and high photothermal transduction.

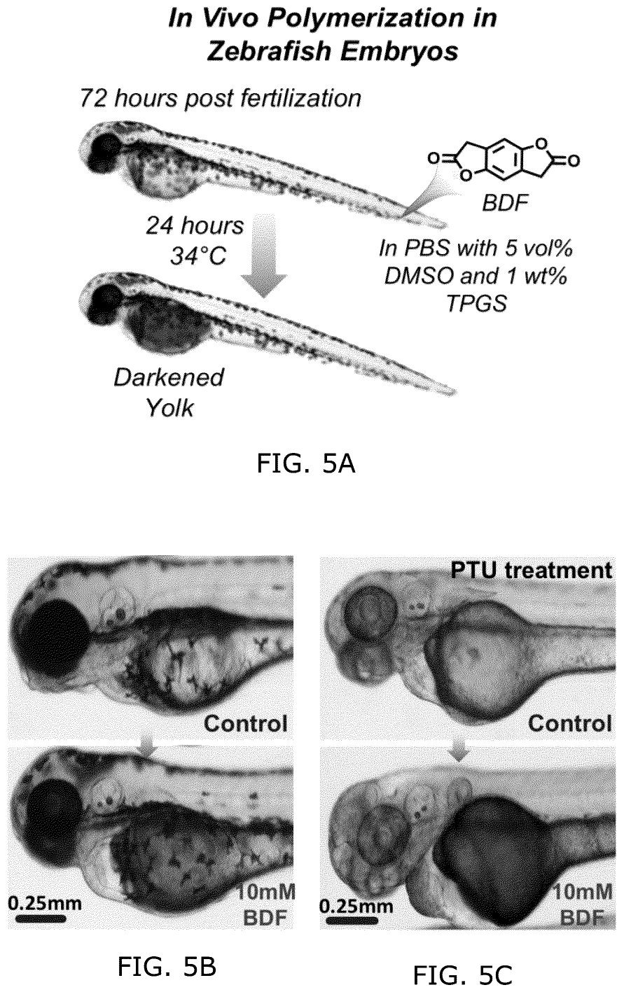

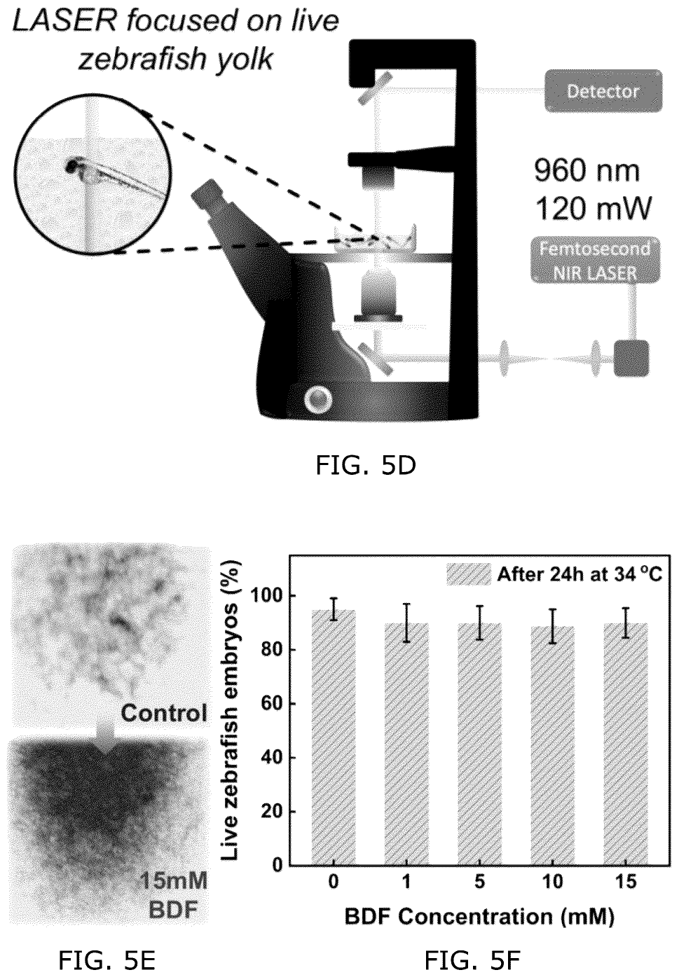

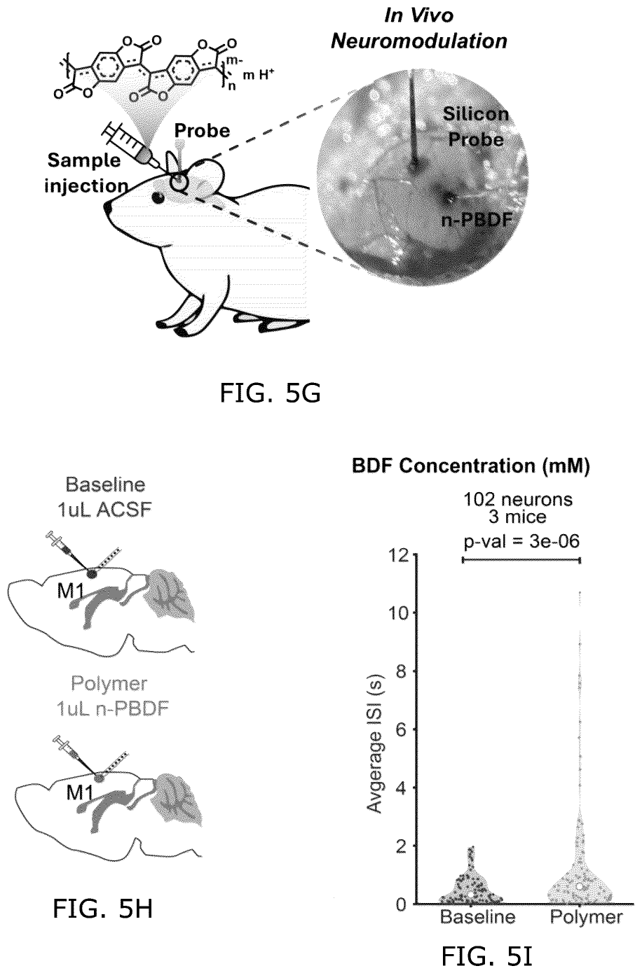

Finally, BDF monomer with 1% w/w TPGS in the PBS media solution was injected in the vasculature of 3-day post-fertilization zebrafish embryos to validate the whole blood catalyzed in vivo polymerization of BDF to synthesize n-PBDF. The fish embryos were injected at the end of the tail (FIG. 5A) with different BDF concentrations from 1 mM to 15 mM and kept inside an incubator at 34° C. Only the 5 mM concentration and above resulted in the darkening of the yolk after 24 hours (FIG. 5B). These results matched the minimum BDF concentration required for efficient n-PBDF polymerization using whole blood as the catalyst. To prevent the misconception of pigmentation in the skin of the embryos as a darkened yolk, zebrafish embryos were treated with 1-phenyl 2-thiourea (PTU). PTU treatment removed the pigment from the skin, allowing for better visualization of the darkening of the embryo's yolk (FIG. 5C). In the case of a 1 mM BDF concentration injection, a clear yolk was observed similar to that seen in control zebrafish embryos. Significant darkening happened when the concentration was increased to 10 mM. Furthermore, 15 mM BDF concentration was injected into the zebrafish embryo, where the yolk was even darker with respect to the 10 mM BDF concentration injection. To characterize the darkened yolk, UV-Vis-NIR measurements were collected on all zebrafish embryos with different concentrations of BDF injections along with the control fish (only PBS injection). When a 15 mM BDF concentration was injected into the zebrafish embryo, a noticeable peak at 960 nm was observed after 24 hours of incubation, indicating the presence of an un-doped PBDF and providing some indication of the in-vivo polymer formation from the BDF monomer. Only in the case of 15 mM BDF injection was the PBDF peak prominent, while in the case of 10 mM injection, the PBDF peak, although present, was overshadowed by the broadening of the peak while overlapping with the peak at 840 nm. To further verify the formation of PBDF, a 960 nm NIR femtosecond LASER focusing was used on the yolk of the incubated zebrafish embryos (FIG. 5D). The absorption images showed higher absorption in the case of 15 mM BDF-injected embryos than the control zebrafish embryos (FIG. 5E). It was noted that BDF injection and in vivo polymerization of PBDF inside zebrafish embryos showed almost no toxicity (FIG. 5F), with at least 80% of the embryos alive after 24 h incubation and showing movement similar to the control embryos. Upon close examination, the beating heart of the embryos was confirmed under a microscope, for both without and with PTU-treated zebrafish embryos.

Because the BDF monomer can lyse the RBCs, the cytotoxicity of the BDF monomer and n-PBDF in A549 lung cancer cells was further studied. It was observed that the n-PBDF polymer is non-toxic to cancer cells, with cell viability remaining close to 100% regardless of the polymer concentration. However, as the concentration of monomer increased from 5 μg/mL to 0.167 mg/mL, the viability of cells decreased significantly from approximately 75% to around 10%, respectively. This indicated that the monomer is toxic to cancerous cells, as demonstrated by a 72-hour assay. Similarly, on the 6-hour assay, similar results were seen with slightly improved cell viability at higher concentrations of BDF monomer. Although the BDF monomer is toxic in cancer cells, it may not impede the in vivo polymerization process, as once the polymerization reaction begins, the toxicity quickly diminishes.

The neural activities of head-fixed locomoting mice were evaluated upon injection of an n-PBDF solution synthesized in ACSF in vivo (FIG. 5G). Using a silicon probe, the spiking of putative single units was compared under baseline and injection of 1 μL n-PBDF solution in the primary motor cortex (M1) (FIG. 5H). It was observed that the average inter-spike interval (ISI) increased from 0.51 to 1.34 seconds after n-PBDF injection (FIG. 5I, n=102 neurons from 3 mice, Wilcoxon signed rank test, p<0.001), suggesting the potential of using n-PBDF as a non-transgenic neural modulation approach in vivo. Notably, the injection of n-PBDF did not alter features in the local field potential (LFP) profile, such as the approximately 40 Hz gamma oscillations, which are critical hallmarks of interaction between cortical inhibitory and excitatory neurons, suggesting the negligible impact of n-PBDF injection on local excitation/inhibition balance. Moreover, the n-PBDF polymer remained in the injection site for extended periods without gradually diffusing to other brain areas. This highlighted local suppression of neuronal excitability in live animals using a biocompatible n-type conductive polymer, n-PBDF, which holds promise as a neuromodulation tool and biomedical treatment of numerous neurological disorders accompanied by abnormal neural excitability, such as epilepsy and Parkinson's disease.

Based on the above, the investigations demonstrated that the conductive polymer n-PBDF can be successfully synthesized from its precursor monomer BDF within biological systems, including living systems, utilizing endogenous proteins as catalysts for the polymerization process, and n-PBDF formed in this manner is capable of exhibiting effective neural activation within biological systems. Coupled with its simplicity and superior biocompatibility, the ability to polymerize and conductive polymer within living organisms opens up promising prospects for its application in future biomedical innovations.

From the above, it can be appreciated that a method is provided for synthesizing the conductive polymer n-PBDF within biological systems utilizing an endogenous catalyst. The method includes providing a biological system in which an endogenous catalyst is present, placing a quantity of BDF monomer dissolved in a solvent into the biological system, and reacting the BDF monomer with the endogenous catalyst to synthesize the n-PBDF conductive polymer within the biological system. The endogenous catalyst may be hemin, a hemoprotein, red blood cells, and whole blood. Notable examples of hemoproteins that can be utilized include hemoglobin, myoglobin, cytochrome, catalase, and peroxidase. The biological system may be a nonliving organic system (such as a neuron) or a living system. Though the in vivo investigations were limited to experiments with zebrafish, the results also establish efficacy in other living systems, including but not limited to human beings, livestock, and other living systems in which hemin, hemoproteins, and/or blood is present. The solvent may be an aqueous solvent such as water, phosphate buffer solution (PBS), or a RPMI-containing media solution. The n-PBDF produced as described above can be used in biomedical applications, as non-limiting examples, bioimaging, drug delivery, biosensors, neural stimulation, and neural modulation.

While the present disclosure has been described with reference to certain embodiments and investigations, it will be apparent to those of ordinary skill in the art that other embodiments and implementations are possible that are within the scope of the present disclosure without departing from the spirit and scope of the present disclosure. Thus, the implementations should not be limited to the particular limitations and investigations described.

1. A method of synthesizing n-doped poly(3,7-dihydrobenzo[1,2-b:4,5-b′]difuran-2,6-dione (n-PBDF) conductive polymer, the method comprising:
providing a biological system in which an endogenous catalyst is present;
placing a quantity of 3,7-dihydrobenzo[1,2-b:4,5-b′]difuran-2,6-dione (BDF) monomer dissolved in a solvent into the biological system; and
reacting the BDF monomer with the endogenous catalyst to synthesize the n-PBDF conductive polymer within the biological system.

2. The method of claim 1, wherein the endogenous catalyst contains iron.

3. The method of claim 1, wherein the endogenous catalyst is chosen from the group consisting of hemin, a hemoprotein, red blood cells, and whole blood.

4. The method of claim 1, wherein the endogenous catalyst is a hemoprotein chosen from the group consisting of hemoglobin, myoglobin, cytochrome, catalase, and peroxidase.

5. The method of claim 1, wherein the endogenous catalyst is a hemoprotein that is present in an amount of at least 0.1 mol %.

6. The method of claim 1, wherein the biological system is a living system or a non-living organic system.

7. The method of claim 1, where in the biological system is a living system.

8. The method of claim 1, where in the solvent is an aqueous media solution.

9. The method of claim 8, wherein the aqueous media solution is a phosphate buffer solution (PBS) or an RPMI-containing media solution.

10. The method of claim 1, wherein the BDF monomer is placed into the biological system at a neuron of the biological system.

11. The method of claim 1, wherein the n-PBDF conductive polymer is near a cortical pyramidal neuron.

12. The method of claim 11, wherein the n-PBDF conductive polymer is at an apical dendrite of a cortical pyramidal neuron.

13. A method of using the n-PBDF conductive polymer synthesized by claim 1, the method comprising using the n-PBDF conductive polymer in a biomedical application.

14. The method of claim 13, wherein the biomedical application is chosen from the group consisting of bioimaging, drug delivery, biosensors, neural stimulation, and neural modulation.

15. The method of claim 13, wherein the biomedical application comprises modulation of neural excitability.

16. The method of claim 15, wherein the modulation is light-induced modulation.

17. The method of claim 16, wherein the light-induced modulation utilizes light in the ultraviolet, visible and near infrared wavelength spectrum.

18. The method of claim 13, wherein the n-PBDF conductive polymer is near a cortical pyramidal neuron.

19. The method of claim 18, wherein the n-PBDF conductive polymer is at an apical dendrite of a cortical pyramidal neuron.

20. A method comprising:
synthesizing n-PBDF conductive polymer in vivo within a biological system by reacting BDF with an endogenous catalyst; and
modulating neural excitability of neurons within the biological system using light-induced modulation of the n-PBDF conductive polymer.
